(19) United States
(12) Patent Application Publication
(10) Pub. No.: US 2011/0217290 A1
(43) Pub. Date: Sep. 8, 2011

(54) TREATMENT AND/OR PREVENTION OF PEANUT INDUCED ANAPHYLAXIS

(76) Inventors: Manel Jordana, Hamilton (CA); Susan Waserman, Toronto (CA); Katherine Arias, Hamilton (CA)

(21) Appl. No.: 13/059,787

(22) PCT Filed: Aug. 18, 2009
(86) PCT No.: PCT/CA2009/001177

§ 371 Date: May 13, 2011

(51) Int. Cl. A61K 39/395; C07K 2/00; C07H 21/04; A61K 38/02; A61K 31/7088; C07D 471/04; C07K 16/00; A61K 31/713; A61K 31/4402; A61K 31/437; A61P 37/08

(52) U.S. Cl. 424/130.1; 530/350; 536/23.1; 514/1.1; 514/44 R; 546/118; 530/387.1; 536/24.5; 514/44 A; 514/352; 514/303

(57) ABSTRACT

Compositions and methods for the treatment of food allergies, particularly peanut allergy are provided. The compositions and methods are based on the observation that agents which interfere with the association of platelet activating fact (PAF) with its receptor (PAF-R) significantly reduce the severity and duration of anaphylaxis. Combination therapy with a PAF inhibitor and a histamine inhibitor provided an increased beneficial effect compared to treatment alone.

### FIELD OF INVENTION

The present invention relates to methods and compositions for the treatment and/or prevention of anaphylaxis.

### BACKGROUND OF THE INVENTION

Immediate hypersensitivity reactions to foods account for one-third to one-half of anaphylaxis cases in emergency departments worldwide. Recent studies in North America and the United Kingdom have reported that the prevalence rates of peanut allergy among schoolchildren is currently over 1%, and this allergy is therefore a major health concern. Anaphylaxis is an acute and life-threatening systemic allergic reaction, and food anaphylaxis fatality registries in the US implicate peanuts as a major culprit of fatal anaphylactic reactions. While other food allergies generally resolve during the first 5 years of age, peanut hypersensitivity usually persists. Thus, peanut allergy is typically life-long, often severe, and potentially fatal.

In contrast to other allergic diseases, prophylaxis and treatment for food allergies such as peanut allergy have advanced very little in recent years, and intervention remains limited to strict avoidance. However, accidental ingestion is common. In addition, at least 87% of patients experiencing food-related fatalities (90% triggered by peanut) had a prior history of a reaction to the responsible food allergen. Epinephrine is the primary treatment. However, timely administration does not always occur due to the lack of immediate accessibility or awareness of the diagnostic criteria.

Mechanistically, anaphylaxis is defined as a hypersensitivity reaction involving the massive release of mediators from mast cells and basophils following allergen interaction with cell-bound immunoglobulin E (IgE). Mediators include vasoactive amines (e.g., histamine and serotonin), proteases and lipid-derived mediators such as leukotrienes, prostaglandins and platelet-activating factor (PAF). In general, these mediators act on target cells to increase smooth muscle contraction, mucin secretion, vascular permeability and vasodilation. Leading to laryngeal edema, acute airway obstruction, hypotension, heart rate alteration, vascular leakage and hypothermia. It is thought that the extent of mediator release closely correlates with the severity and persistence of the anaphylactic reaction. However, the relative contribution of these mediators in the physiopathology of food-induced anaphylaxis is unknown.

### SUMMARY OF THE INVENTION

The impact of pharmacological interventions targeting either metabolic pathways or mediator receptors in an experimental mouse model of peanut-induced anaphylaxis (PIA) was examined. The present invention demonstrates that blockade of platelet activating factor (PAF) activity significantly reduces prolonged and life-threatening peanut-induced anaphylactic reactions. Furthermore, combination therapy targeting PAF and histamine receptors has a synergistic effect in the prevention of peanut-induced anaphylactic responses. The present invention demonstrates that the concurrent blockade of PAF and histamine provides a novel life-saving therapeutic approach for food-induced anaphylaxis.

A pharmaceutical composition for the prevention or treatment of a food allergy comprising an agent that reduces PAF activity.

The agent is preferably a compound that inhibits interaction of platelet activating factor (PAF) with its receptor (PAF-R) and a pharmaceutically acceptable carrier.

In another embodiment, the agent is selected from the group consisting of a small molecule, polypeptide and nucleic acid.

A composition wherein the agent is a PAF-R antagonist.

A composition wherein the PAF-R antagonist is ABT491.

A composition wherein the polypeptide is an antibody or a fragment thereof.

A composition wherein the nucleic acid is an anti-sense oligonucleotide, dsRNA, siRNA, shRNA and the like.

A composition wherein the composition further includes an antihistamine.

A composition wherein the antihistamine is a H1 or H2 receptor antagonist.

A composition according to claim 9 wherein the antihistamine is mopyramine maleate.

A composition wherein the food allergy is a peanut allergy.

A method of preventing or treating anaphylaxis comprising administering an agent that alters PAF activity in a manner and amount to reduce PAF activity.

A method wherein the agent is a compound that inhibits interaction of platelet activating factor (PAF) with its receptor (PAF-R) and a pharmaceutically acceptable carrier.

A method wherein the agonist is administered in a manner selected from the group consisting of oral, mucosal, intraperitoneal, intravenous, sub-cutaneous, intra muscular and organ-specific routes.

The present invention also provides for the use of a PAF inhibitor as an agent as an agent for the treatment of a food allergy, particularly a peanut allergy.

In a preferred embodiment, a composition comprising a PAF inhibitor and a histamine inhibitor is used for the treatment of a food allergy.

### BRIEF DESCRIPTION OF THE DRAWINGS

These and other features of the invention will become more apparent from the following description in which reference is made to the appended drawings wherein:

FIGS. 1A to 1F demonstrate various physiological effects associated with peanut induced anaphylaxis (PIA);

FIGS. 2A to 2H demonstrate the difference in several parameters in control mice, untreated mice and mice treated with various treatments.

### DETAILED DESCRIPTION

PAF is a potent phospholipid secreted by mast cells, monocytes and fixed tissue macrophages. By binding to a unique G protein-coupled seven transmembrane receptor (PAF-R), PAF mediates cellular responses including Ca²⁺ mobilization, priming of superoxide generation, platelet aggregation and vasodilatation. In addition to its role as a physiological mediator, PAF has been associated with the pathogenesis of anaphylactic shock. Administration of PAF to mice can lead to bronchoconstriction, hypotension and increased vascular permeability causing pulmonary edema and impaired cardiac and renal function.

The present invention provides methods and compositions for the treatment of food allergies, particularly peanut allergy, that comprise administering an agent that reduces PAF activity either directly or by inhibiting its interaction with its receptor (PAF-R). The agent may be administered alone or in combination with an agent that inhibits the release of histamine such as H1 and/or H2 receptor antagonists. The PAF activity reducing agent may also be combined with other known allergy treatments, such as conventional immunotherapy or anti IgE treatment.

Various types of inhibitory agents, such as small molecules, polypeptides and polynucleotides may be useful in the practice of the invention. The present invention further encompasses antibodies or fragments thereof that block PAF activity. Polynucleotides, polypeptides, antibodies, and/or agonists or antagonists of the present invention may be useful in treating, preventing, diagnosing and/or prognosing diseases, disorders, and/or conditions of the immune system, particularly allergies, more particularly food allergies such as peanut allergy.

Compositions containing the polypeptides of the invention may be delivered in a variety of ways (e.g., compositions containing polypeptides or polypeptide antibodies associated with heterologous polypeptides, heterologous nucleic acids, toxins, or prodrugs) to targeted cells expressing the polypeptide. Polypeptides or polypeptide antibodies of the invention may be associated with heterologous polypeptides, heterologous nucleic acids, toxins, or prodrugs via hydrophobic, hydrophilic, ionic and/or covalent interactions.

Anaphylactic shock in a subject can be treated by administering to the subject a compound capable of preventing binding of PAF to its receptor PAF-R. The administration occurs for a time and in an amount sufficient to treat or prevent anaphylaxis in a subject.

A synergistic, highly effective method of treating or preventing anaphylaxis in a subject comprises administering a composition comprising a PAF-R antagonist in combination with an antihistamine, for a time and in an amount sufficient to treat or prevent anaphylaxis.

The present invention also provides a method of identifying a compound that ameliorates anaphylaxis. The method involves contacting a cell that expresses the PAF-R with a candidate compound, and comparing the effect in the cell contacted by the candidate compound with the effect in a control cell not contacted by the candidate compound. Physiological parameters such as CA2+ mobilization, superoxide generation, platelet aggregation and/or vasodilation can also be assessed in an animal model of peanut induced anaphylaxis to screen potential antagonists.

Experimental studies associated with the present invention demonstrate that treatment with a PAF-R antagonist significantly diminishes the severity of peanut-induced anaphylaxis and accelerates the recovery from anaphylactic reactions. In addition, a treatment involving administration of a composition comprising a combination of a PAF-R antagonist and antihistamines results in dramatic beneficial results.

Experiments demonstrating the effects of inhibiting PAF or PAF-R with or without inhibitors of histamine activity were performed in a mouse model of peanut allergy. The mouse model of peanut allergy is a well accepted model in which animals display symptoms which closely mimic those found in human anaphylaxis.

In exemplary experiments, female C57BL/6 mice (6-8 wk old) were purchased from Charles River Laboratories and housed under a specific pathogen-free environment and maintained on a 12 h light-dark cycle.

To develop a model of Peanut-induced Anaphylaxis (PIA), mice were sensitized with 1 mg of peanut protein along with 10 μg of cholera toxin (List Biological Laboratories, CA, USA) in 100 μl of sterile water via oral gavage once a week for four weeks. Peanut butter (KRAFT) was used as the source of peanut protein (PP). Control mice were given 100 μl of sterile water. Control and sensitized mice were challenged by injecting 5 mg of crude peanut extract (Greer Laboratories, NC, USA) in 500 μl of PBS into the peritoneal cavity one week after the last sensitization.

Mice were carefully monitored for either 40 min or 120 min immediately following challenge. The anaphylactic responses during these intervals were evaluated as follows: a) Symptom Score—Clinical symptoms were assessed using the following scoring system. 0: no clinical symptoms; 1: repetitive mouth/ear scratching and ear canal digging with hind legs; 2: decreased activity, self isolation, puffiness around eyes and/or mouth; 3: Periods of motionless for more than 1 min and lying on stomach; 4: No response to whisker stimuli, reduced or no response to prodding; 5: tremor, convulsion or death. b) Core Body Temperature (T)—Rectal temperature readings were performed every 10 minutes with a rectal probe digital thermometer (VWR, Mississauga, ON, Canada).

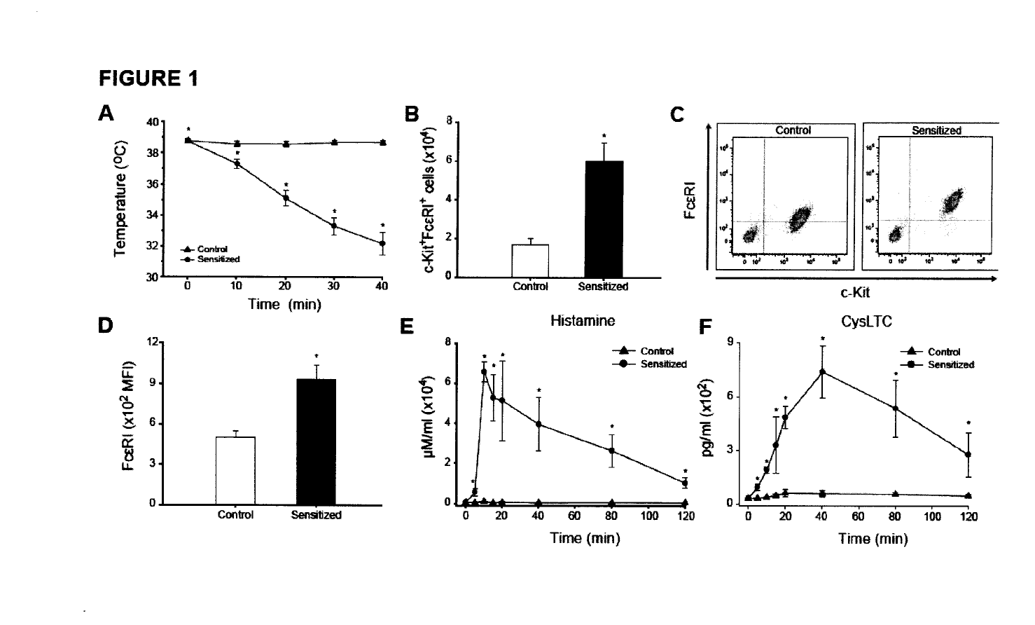

To grade the severity of the anaphylactic response, a 3-point grading scheme was utilized. Accordingly, the anaphylactic reactions were classified as “no reaction” (score 0 and 37.5° C.≦T≦40° C.), “mild reaction” (score ≦2 and 34.5° C.≦T≦37.5° C.) or “severe or life-threatening reaction” (score ≧3 and T≦34.5° C.). The results are shown in FIG. 1.

Peanut-induced anaphylaxis in orally sensitized mice is preceded by up-regulation of FcεRI expression on peritoneal c-kit+ cells and accompanied by release of vasoactive mediators. FIG. 1A shows changes in rectal temperature of control (▴) and sensitized (∘) mice following CPE challenge at the indicated time points. Peritoneal cells were isolated prior to challenge and c-kit+FcεRI+ cells were determined using a LSRII flow cytometer. These results indicate that PIA in this model is characterized by acute, consistent and robust anaphylactic responses as indicated by severe clinical symptoms and a significant drop in core body temperature.

FIG. 1B shows absolute number of cells expressing surface IgE receptor (FcεRI) and c-kit. Sensitization with peanut resulted in a 3-fold increase in the number of peritoneal c-kit+FcεRI+ cells (FIGS. 1 B and C) along with significant up-regulation of surface FcεRI expression (Mean Fluorescence Intensity (MFI) naive: 600 vs sensitized: 900, FIG. 1 D). FIG. 1C is a representative FACS plots gated on CD11b cells are shown for control and sensitized mice. FIG. 1D shows surface levels of FcεRI on c-kit+ cells are expressed as mean values of fluorescence intensity. The number of oral sensitizations progressively increases the level of circulating peanut-specific IgE. Since IgE is a major regulator of mast cell FcεRI expression, increased levels of peanut-specific IgE resulted in greater FcεRI density and occupancy. This is significant since previous studies have shown that up-regulation of FcεRI expression sensitizes MC with a larger number of IgE molecules, thus decreasing the threshold for degranulation and, consequently, amplifying the release of vasoactive and proinflammatory mediators. These results demonstrate that the elicitation of PIA is mediated, to a large extent, by the so-called classical pathway of anaphylaxis, which involves mast cells, IgE and FcεRI (IgE high-affinity receptor)

As shown in FIGS. 1 E and F, significantly greater levels of histamine and cysteinyl leukotrienes (CysLTC) were detected in the plasma of peanut-sensitized mice following CPE challenge compared to control mice. Blood from control and sensitized mice was collected in K2-EDTA containing tubes at the indicated time points following i.p challenge with CPE and plasma was analyzed for (E) histamine and (F) CysLTC levels. Histamine and leukotriene levels were determined in plasma at different time points following challenge using enzyme immunoassay kits (Beckman Coulter Canada, Inc, Mississauga, and Cedarlane Laboratories Ltd, Hornby, Canada, respectively) according to the manufacture's specifications. Plasma was obtained using K₂-EDTA containing tubes (BD Biosciences, Mississauga, ON, Canada). Graphs show mean values±SEM from three to five independent experiments; n=8-12 mice per group. * P<0.05 relative to control. The kinetics of mediator production is distinct. While histamine peaked early after challenge (10 minutes), remained substantially elevated for 1 h and slowly decreased over time, leukotrienes progressively increased upon challenge reaching maximum levels at 40 minutes and gradually declined within 2 to 3 hours following challenge (FIGS. 1 E and F). Since it was observed that mast cell deficient (kitW/kitW-v) mice do not experience any measurable peanut-induced anaphylactic reactions, the results suggest that these mediators are primarily produced, at least in this model, by mast cells. These findings indicate that oral sensitization with peanut results in a significant increase in the number of peritoneal mast cells and FcεRI surface expression likely contributing to the massive release of vasoactive mediators following challenge with peanut.

Results suggest that histamine and leukotrienes are non-essential mediators but are biomarkers of peanut-induced anaphylactic reactions. Several lines of evidence indicate that the release of vasoactive mediators is associated with the clinical manifestations of anyaphylaxis. Nevertheless, the relative contribution, if any, of these mediators in the pathogenesis of food-induced anaphylaxis is not well understood. The development of clinical signs and symptoms of anaphylaxis was accompanied by a marked release of CysLTC and histamine. The physiological involvement of these agents in the process was examined.

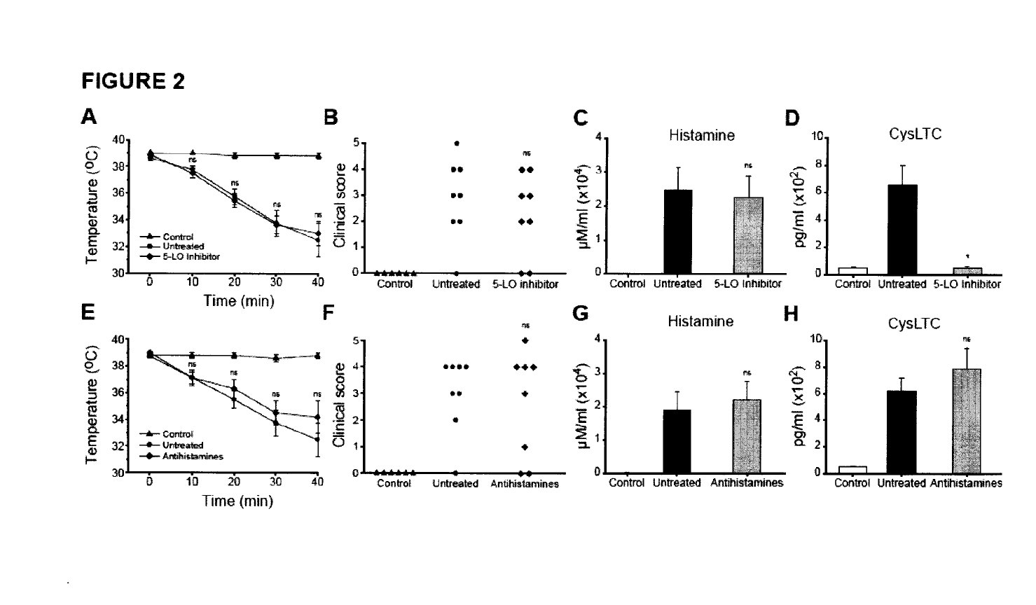

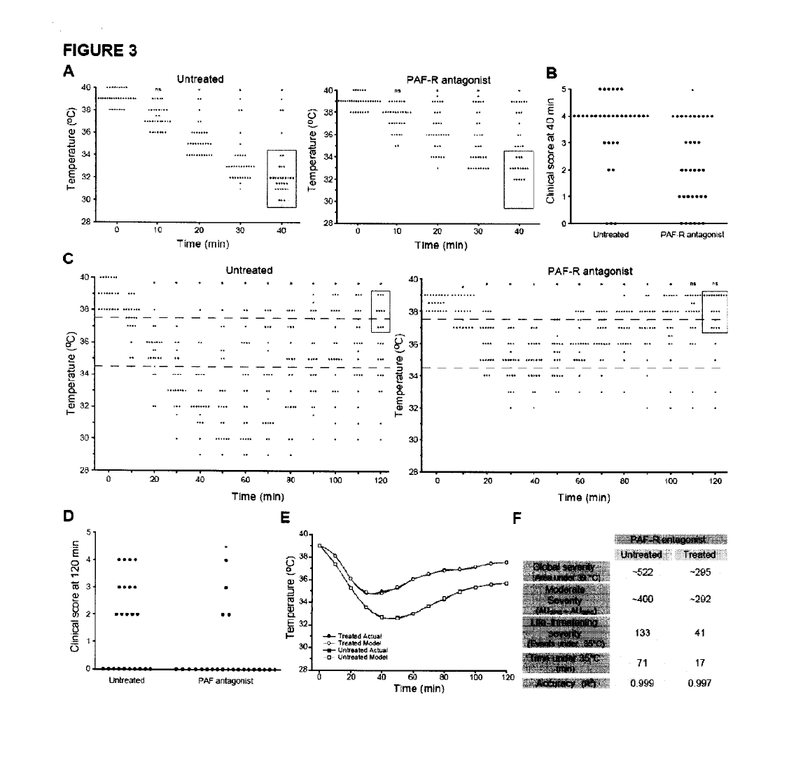

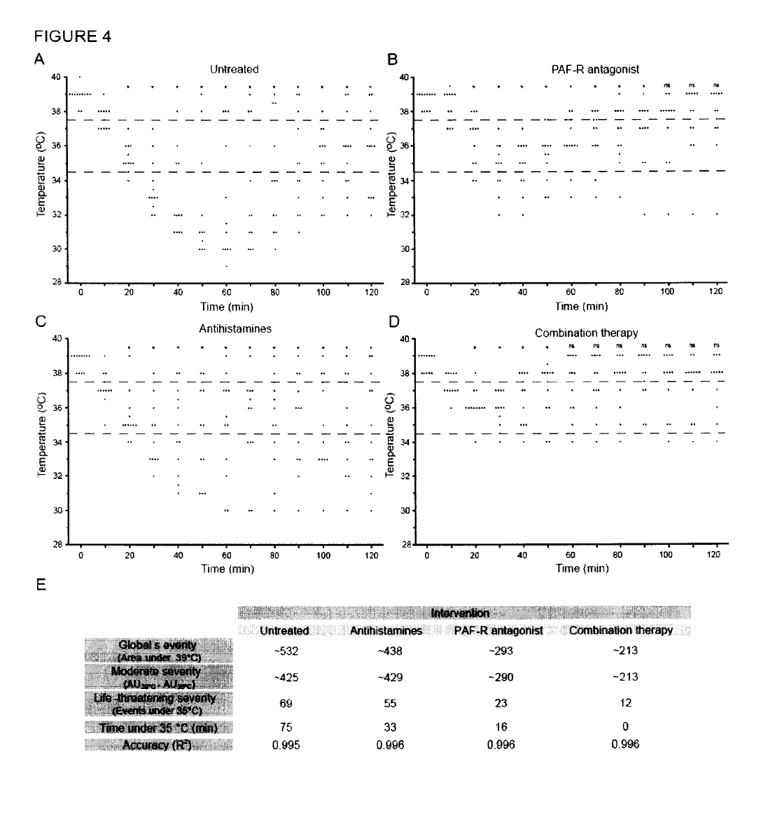

To assess the role of leukotrienes in PIA, peanut-sensitized mice were administered a 5-lipoxygenase (5-LO) inhibitor, Zileuton [50 mg/Kg], via oral gavage twice, 12 h and 1 h, prior to challenge. The results of blockade of histamine and leukotrienes are shown in FIG. 2. Mice were sensitized with 1 mg of peanut protein along with 10 μg of cholera toxin via oral gavage once a week for four weeks. (A-D) One week after the last sensitization, mice were treated with Zileuton (5-LO inhibitor) or administered drug vehicle, 0.5% hydroxyethyl cellulose, (untreated) twice, 12 and 1 h, prior to i.p. challenge with CPE. (E-H) One week after the last sensitization, mice were injected with either PBS (untreated) or histamine receptor antagonists mepyramine [3 mg/kg] and cimetadine [10 mg/Kg] (for H1 receptor and H2 receptor, respectively) i.v. 30 minutes prior to CPE challenge. Control mice were administered 100 μl of sterile water orally once a week for four weeks and challenged one week following the last gavage. These mice did not receive any treatment prior to challenge. (A) and (E) show mean changes in rectal temperature and (B) and (F) show clinical symptoms for individual mice from control (A), untreated (▴) and treated (∘) groups at 40 min following challenge. Histamine (C) and (G) and leukotriene (D) and (H) levels from each of the experimental groups above were analyzed in plasma 40 min following CPE challenge. Data are representative from three independent experiments. *, P<0.05 relative to control mice. ns, not significant. As shown in FIGS. 2 A and B, blockade of the 5-LO pathway did not have an impact on the extent and/or kinetics of peanut-induced anaphylactic reactions. Moreover, we observed comparable levels of histamine in plasma from untreated vs. treated mice (FIG. 2 C) were observed. Conversely, leukotrienes were nearly undetected in plasma from treated, but not in untreated, mice indicating that pharmacological inhibition of the 5-LO pathway was successfully accomplished (FIG. 2 D). In agreement with these findings, mice with a targeted disruption in the 5-LO gene manifested the same degree of anaphylaxis as their wild-type counterparts following challenge (data not shown).

These findings do not negate the potential value of leukotriene levels in the management of anaphylaxis. Indeed, laboratory tests to support the clinical suspicion of anaphylaxis have been rather disappointing to date. Transiently elevated plasma histamine levels of >10 nM correlate with the severity and persistence of cardiopulmonary or gastrointestinal manifestations. However, histamine peaks within few minutes of onset of anaphylaxis symptoms and has a very short half-life; thus, elevations are transient and can be often missed. Identification of an elevated serum tryptase level (>11.4 ng/ml) within 12 h (preferably within 3 h) of the onset of an episode is more widely used as a confirmatory test. However, total serum tryptase levels within the normal range have been found in patients with clinically confirmed food-induced anaphylaxis, thus suggesting that it is a poor biomarker for diagnosis. Recently, the usefulness of mediators such as carboxypeptidase A3, PAF and PAF-AH to support the clinical diagnosis of anaphylaxis is being explored. Leukotriene levels, albeit not involved in the elicitation of acute and systemic anaphylactic reactions, gradually increase in plasma following peanut challenge and remain elevated for a considerable period of time, thus suggesting that they may represent a useful biomarker of anaphylaxis. In this regard, the presence of leukotrienes and their metabolites has been previously reported in body fluids (i.e. urine) during and following anaphylaxis.

Previous studies have documented the role of histamine in the regulation of core body temperature and respiratory function in passive systemic anaphylaxis. However, the contribution of histamine to the clinical manifestations of active systemic anaphylaxis has been poorly investigated. To examine the role of histamine on PIA, peanut-sensitized mice were administered H1 and H2 receptor antagonists (Mepyramine maleate [3 mg/Kg] and Cimetidine [15 mg/Kg], respectively) intravenously 30 min prior to challenge. The data indicate that blocking H1 and H2 receptors did not change the severity or the course of the anaphylactic reaction (FIGS. 2 E and F). Moreover, we observed comparable levels of histamine and CysLTC (FIGS. 2 G and H) in plasma between treated vs. untreated mice following peanut challenge were observed suggesting that the extent of mast cell degranulation was equivalent in both groups.

Presently, H1- and H2-antihistamines are often given as adjuvant therapies. However, the benefit of antihistamines in systemic anaphylaxis has not been conclusively assessed. Compared to adrenaline, the initial treatment of choice for anaphylaxis, antihistamines have a slow onset of action and cannot block events that occur subsequent to histamine binding to its receptors. Since plasma histamine peaks early in anaphylaxis and rapidly returns to baseline, it is thought that the timing of administration of antihistamines is the main factor limiting their effectiveness. In the present study, H1 and H2 antagonists were administered prior to peanut challenge; however, their impact on clinical signs such as a drop in core body temperature was negligible. Thus, the present findings provide conclusive evidence that antihistamines, at least when given alone, do not ameliorate the clinical manifestations of PIA.

There is accumulating evidence linking PAF to the pathology of anaphylaxis and endotoxic shock in animals. In fact, various anaphylactic symptoms can be mimicked by PAF injection in animals. Importantly, Vadas et al. have recently shown that circulating PAF levels correlate with the severity of anaphylactic reactions triggered by foods, medications or insect stings in humans. However, causality between PAF levels and severity of anaphylaxis could not, understandably, be ascertained in this study. Peanut-sensitized mice were treated with a PAF-R antagonist [50 mg/Kg] orally lh prior to challenge and carefully monitored them for 40 minutes. FIG. 3 illustrates that treatment with a PAF-R antagonist substantially diminishes the severity of peanut-induced anaphylaxis and accelerates the recovery from anaphylactic reactions. Mice received either PBS (untreated) or a PAF-R antagonist, ABT-491[50 mg/Kg] orally 1 h prior to i.p. challenge with 5 mg of CPE (FIG. 3).

Targeting PAF activity considerably lessened the severity of anaphylactic reactions (FIGS. 3 A and B). Approximately, 50% of treated mice developed either no (score 0 and 37.5° C.≦T≦40° C.) or mild (score ≦2 and 34.5° C.≦T≦37.5° C.) anaphylactic reactions following challenge with peanut. Notably, there was a considerable difference in the number of mice that dropped their core temperature by more than 4° C. (87% in untreated vs. 48% in treated) suggesting that PAF antagonism considerably prevented life-threatening (score ≧3 and T≦34.5° C.) reactions. Moreover, the treatment completely prevented the development of seizures or death. While 15% of untreated mice developed convulsions, no seizures were observed in the treated group. It is important to note that the levels of peanut-specific IgE and IgG₁ were similar among groups indicating equivalent sensitization (data not shown). The data demonstrate that the blockade of PAF-R substantially prevents severe anaphylactic reactions to peanuts.

To investigate whether PAF-R antagonism had an impact on the time of recovery from an anaphylactic reaction, clinical responses were monitored for 120 min following peanut challenge, instead of the usual 40 min. Blocking the biological activity of PAF significantly accelerated recovery (FIGS. 3 C and D). There was a clear difference in the number of mice that reached recovery temperature (37° C.) within 120 min following challenge (83% in treated vs. 43% in untreated; FIG. 3 C). It should be noted that mice experiencing prolonged severe reactions (score ≧3 and T≦34.5° C.) showed a slower rate of recovery and, in some cases, did not show any clinical improvement during the observation period. Approximately 30% of untreated, but only 9% of treated, mice experienced severe hypothermia 120 min following challenge, thus, decreasing their likelihood to fully recover and, correspondingly, increasing the probability of death. Approximately 20% of untreated, but none of the treated, mice died within 48 h following challenge. Collectively, the data provide evidence that PAF-R antagonism markedly attenuates the magnitude and duration of peanut-induced anaphylactic reactions.

In an effort to more comprehensively grade the anaphylactic response, three measures of severitywere utilized: global, moderate and life-threatening severity. To facilitate the assessment of severity, a mathematical model that describes the overall behavior of the anaphylactic response for each experimental group was developed. Using mean experimental data for untreated mice, an equation was derived that represents the pattern in core temperature up to 120 minutes following challenge. The same equation was then fit to the other experimental group (mice treated with PAF-R antagonist) to derive a numerical coefficient that accurately depicts the respective response. (FIG. 3 E). These equations were subsequently used to generate modeled data. Actual and modeled data were validated through linear regression, yielding R² values of greater than 0.99 for both untreated and treated groups (FIG. 3 F). Thus, the response for each experimental group can be expressed with a mathematical representation that can be used to assess the overall degree of the anaphylactic response. Global severity was defined as the area of the response under 39° C. (AU_{39° C.}), while life-threatening severity was depicted as the area of the response under 35° C. (AU_{35° C.}; FIG. 3 F). Moderate severity was deemed as the difference between global and life-threatening severity (AU_{39° C.}-AU_{35° C.}; FIG. 3 F). Given that these areas (i.e. AU_{39° C.} and AU_{35° C.}) were calculated using mean temperatures at each time point, they describe the overall trend of the response. Hence, the measure of AU_{35° C.} does not differentiate life-threatening conditions (T≦34.5° C.) from the overall response. In other words, the AU_{35° C.} is not representative of those mice that experienced life-threatening anaphylaxis exclusively but rather encompasses the response of the entire population. Therefore, the concept of life-threatening severity was modified to consider only the number of mice whose temperature fell below 35° C. at any time point (expressed as ‘events under 35° C.’). In addition, the duration of life-threatening conditions was determined by calculating the time at which each response was below 35° C. (FIG. 3 F).

The results demonstrate that treatment with a PAF-R antagonist resulted in approximately 50% decrease in global severity. In addition, the number of ‘events under 35° C.’ was 41 in treated mice vs. 133 in untreated mice, indicating that untreated mice approached life-threatening conditions to a much greater extent. This is relevant as mice experiencing a severe drop in core temperature are less likely to recover and, thus, succumb to challenge. Moreover, treated mice were under life-threatening conditions for approximately 75% less time compared to untreated mice (FIG. 3 F).

During the course of a systemic anaphylactic reaction vasodilatation and hypotension are often associated with excessive vascular permeability and fluid extravasations. In accordance with this, it was observed that the blockade of PAF biological activity substantially reduced vascular leakage as indicated by a 57% decrease in the content of albumin in the PL fluid of treated mice compared to untreated mice following peanut challenge. In addition, treatment with the PAF-R antagonist was associated with a significant decrease in the levels of histamine (untreated: 18791.3±5460.6 μM/ml vs. treated: 10261.2±3108.5 μM/ml) and Cysts (untreated: 570.2±237.3 pg/ml vs. treated: 187.3±96.9 pg/ml) in plasma following challenge with peanut. These data are in agreement with in vitro studies showing that PAF can directly induce histamine release and de novo synthesis of leukotriene C4 (LTC4) from various cell types in response to appropriate stimuli. It is, therefore, plausible that counteracting PAF may have resulted in a concurrent decrease in other vasoactive mediators and, thus, a considerable reduction in the extent of the anaphylactic response.

A growing number of studies have illustrated the complexity of the interactions and redundancy among vasoactive mediators involved in allergic disease, and suggested that the control of clinical manifestations may require the blockade of various mediators.

The potential adjuvant effect of antihistamines in the treatment of PIA was assessed. Peanut-sensitized mice were treated with a PAF-R antagonist along with H1 and H2 receptor antagonists and monitored them for 120 min following challenge. FIG. 4 illustrates that concomitant treatment with PAF-R antagonist and anti-histamines prevents life-threatening and prolonged peanut-induced anaphylactic reactions. Mice were sensitized with 1 mg of peanut protein along with 10 μg of cholera toxin via oral gavage once a week for four weeks. One week after the last sensitization, separate groups of mice were treated as follows: (A) appropriate drug vehicle (B) PAF-R antagonist [50 mg/Kg], ABT-491] orally 1 h prior to CPE challenge. (C) histamine receptor antagonists mepyramine [3 mg/kg] and cimetadine [10 mg/Kg] (for H1 receptor and H2 receptor, respectively) i.v. 30 min prior to CPE challenge (D) The last group of mice was administered the treatments described in B and C concurrently. Pooled data from two independent experiments are shown (n=6/experiment). *, P<0.05 relative to time 0. ns, not significant. In order to compare the efficacy of these various interventions, we developed a mathematical model to grade the severity of the anaphylactic response. (E) The accuracy of the model was quantified using linear regression. Using the equation from the model, global severity was defined as the area between the response and 39 (area under 39° C. (AU39° C.)) while life-threatening severity was defined as the area of the response under 35° C. (AU35° C.). Since these areas were calculated using mean temperatures at each time point, they describe the overall trend of the response. Hence, the measure of AU35° C. does not differentiate life-threatening conditions (T≦34.5° C.) from the overall response. In other words, the AU35° C. is not representative of those mice that experienced life-threatening anaphylaxis exclusively but rather encompasses the response of the entire population. The concept of life-threatening severity was modified to consider only the number of mice whose temperature fell below 35° C. at any time point (expressed as ‘events under 35° C.’). The data demonstrate that combination therapy targeting PAF and histamine has a remarkably beneficial effect on treating signs and symptoms of PIA (FIG. 4 A-D). As shown in FIGS. 4 D and E all but one mouse developed mild, if any, anaphylactic reactions indicating that concurrent blockade of PAF and histamine has the greatest beneficial effect in the prevention of peanut-induced anaphylactic reactions.

In order to compare the efficacy of these various interventions, the newly developed mathematical model was used to grade the severity of the anaphylactic response. The model shows that the global response up to 120 min (AU_{39° C.}) in the group treated with the combination therapy never fell below 35° C. (FIG. 4 E). Similarly, the measure of moderate severity reflects the success of the combination therapy in the prevention of severe drop in core body temperature. While PAF antagonism had a partial effect on moderate severity, histamine blockade did not. The number of ‘events under 35° C.’ were 75, 33, 16 and 0 for untreated, antihistamines and PAF-R antagonist groups, respectively indicating that combination therapy was the most successful intervention to prevent a drastic reduction in core temperature and, hence, the development of life-threatening reactions (FIG. 4 E). Similarly, the proportion of mice reaching partial or full recovery at 120 min was greatest when treated with combination therapy. Of note, all of these mice achieved some level of recovery with 66% of them exhibiting full recovery.

Mathematical equations and analysis were generated from the mean experimental data using curve-fitting techniques from FindGraph VERSION 1.821 (UNIPHIZ Lab, Vancouver, BC, Canada). For each experimental group, temperature (T) is expressed as a function of time (t), resulting in the following equation:

T=39+xₐt+x_{b}t²+x_{c}t³+x_{d}t⁴+xₑt⁵+x_{f}t⁶+x_{g}t⁷+xₕt⁸

where xn represents numerical coefficients unique to each individual group, calculated through FindGraph. The calculation of the areas above each curve was derived using Mathematica 6.0.2.1 (Wolfram Research Inc., Champaign, Ill., USA). For each group, response curves were programmed to calculate the definite integral below either 35° or 39° C., for the duration that each curve was below these temperatures. Accuracy of the model was measured through linear regression, comparing actual and simulated data for each group, yielding R2 values.

Data are expressed as mean□SEM. Statistical analysis was performed using SimgaStat™ (Systat Software Inc, San Jose, Calif., USA). When applicable, results were analyzed using one- or two-way ANOVA with repeated measures followed by Tukey post hoc test. Unpaired student t-test (two tailed) was used when only two sets of continuous data were compared. In cases of raw data failing normality, analysis was performed on Ranks using Dunn's method. A p value of □0.05 was considered statistically significant. FIGS. 3 and 4 illustrate in an animal model the effect of treatment with a PAF receptor antagonist either alone or in combination with an antihistamine. The results indicate that treatment with a PAF-R antagonist significantly attenuates the magnitude and duration of anaphylactic reactions. Combined blockade of PAF activation and histamine resulted in a greater beneficial effect. Furthermore, the combination therapy was associated with a significant decrease in vascular leakage and release of vasoactive mediators following challenge.

These findings were made in an experimental model that elicits robust anaphylactic reactions. This indicates that an even a greater benefit may be anticipated in situations of less severe reactions. These findings have implications beyond their inherent, direct, therapeutic value. The evaluation or application of promising strategies such as conventional immunotherapy or anti-IgE treatment is largely limited by the potential of serious adverse effects. On the other hand, the pharmacological methods and compositions of the present invention may be viewed as a risk-reducing therapy. In other words, an ancillary therapy that facilitates the implementation of other therapeutic strategies for peanut allergy and, most likely, other food allergies is provided. These results indicate that treatment with PAF or PAF-R antagonists, particularly combination therapy with antihistamines provides a potent therapeutic modality for the prevention and treatment of food allergies.

While the studies described address the activity of specific agonist and antagonists, the present invention is not limited to the exemplary agents used to demonstrate the utility of the invention. One skilled in the art would recognize that another agent that reduces PAF activity would be useful in the methods of the invention to prevent or treat anaphylaxis. Furthermore, the exemplified methods and compositions could easily be modified to assess the action of polynucleotides or polypeptides of the invention. In addition, the invention is not limited to peanut allergy. Since food allergies typically follow the same pathway, it is likely that the methods and compositions of the invention would be effective for the treatment of other food allergies.

The present invention addresses the need for improved therapeutic and/or preventative methods and compositions for the management of food allergies, particularly peanut allergy. Platelet activating factor (PAF) antagonism significantly attenuates the magnitude and duration of anaphylactic reactions. Combined blockade of PAF and histamine had a greater beneficial effect than either alone. The data presented indicate that combination therapy provides a potentially life-saving therapeutic approach for peanut allergy and likely other food induced anaphylaxis.

1. A pharmaceutical composition for the prevention or treatment of a food allergy comprising an agent that reduces PAF activity.

2. A composition according to claim 1 wherein the agent is a compound that inhibits interaction of platelet activating factor (PAF) with its receptor (PAF-R) and a pharmaceutically acceptable carrier.

3. A composition according to claim 1 wherein the agent is selected from the group consisting of a small molecule, polypeptide and nucleic acid.

4. A composition according to claim 1 wherein the agent is a PAF-R antagonist.

5. A composition according to claim 4 wherein the PAF-R antagonist is ABT491.

6. A composition according to claim 3 wherein the polypeptide is an antibody or a fragment thereof.

7. A composition according to claim 3, wherein the nucleic acid is an anti-sense oligonucleotide, dsRNA, siRNA, shRNA and the like.

8. A composition according to claim 1 wherein the composition further includes an antihistamine.

9. A composition according to claim 8 wherein the antihistamine is a H1 or H2 receptor antagonist.

10. A composition according to claim 9 wherein the antihistamine is mopyramine maleate.

11. A composition according to claim 1 wherein the food allergy is a peanut allergy.

12. A method of preventing or treating anaphylaxis comprising administering an agent that alters PAF activity in a manner and amount to reduce PAF activity.

13. A method according to claim 12 wherein the agent is a compound that inhibits interaction of platelet activating factor (PAF) with its receptor (PAF-R) and a pharmaceutically acceptable carrier.

14. A method according to claim 13 wherein the agonist is administered in a manner selected from the group consisting of oral, mucosal, intraperitoneal, intravenous, sub-cutaneous, intra muscular and organ-specific routes.

15. Use of a composition as defined in claim 1 for the treatment of a food allergy.

16. Use according to claim 15 wherein the food allergy is peanut allergy.
